SEQUENCE LISTING (8 sequences)

SEQ ID NO: 1 - protein, 5 aa, Homo sapiens
NYGIT

SEQ ID NO: 2 - protein, 17 aa, Homo sapiens
WISAYNGNTNYAQKLQD

SEQ ID NO: 3 - protein, 15 aa, Homo sapiens
DRVPRIPVTTEAFDI

SEQ ID NO: 4 - protein, 13 aa, Homo sapiens
SGSSSNIGSYFVY

SEQ ID NO: 5 - protein, 7 aa, Homo sapiens
RNNQRPS

SEQ ID NO: 6 - protein, 12 aa, Homo sapiens
AAWDDSLSGHWV

SEQ ID NO: 7 - protein, 126 aa, Homo sapiens
QVQLVQSGAEVKKPGASVKVSCKASGTYRTFTNYGITWVRQAPGQGPEWMGWISAYNGNTNYAQKLQDRVTVTTDTSTSTAYMELRSLRSDDTAVYYCARDRVPRIPVTTEAFDIWGQGTMVTVSS

SEQ ID NO: 8 - protein, 113 aa, Homo sapiens
QSVLTQPPSASGTPGQRVTISCYSSGSSSNIGSYFVYWYQQLPGTAPKLLIYRNNQRPSGVPDRFSGSESGTSASLAISGLRSEDEADYYCAAWDDSLSGHWVFGGGTKLTVL

(19) United States
(12) Patent Application Publication
(10) Pub. No.: US 2015/0005475 A1
(43) Pub. Date: Jan. 1, 2015

(54) PROCESS FOR REDUCING ANTIBODY AGGREGATE LEVELS AND ANTIBODIES PRODUCED THEREBY

(71) Applicant: MEDIMMUNE LIMITED, Cambridge (GB)

(72) Inventors: Justyna Kucia, Cambridge (GB); Marcel Kuiper, Cambridge (GB); Richard Tran, Cambridge (GB); David Gruber, Cambridge (GB)

(73) Assignee: MEDIMMUNE LIMITED, Cambridge (GB)

(21) Appl. No.: 14/375,244

(22) PCT Filed: Feb. 1, 2013
(86) PCT No.: PCT/EP2013/052078

§ 371 Date: Jul. 29, 2014

Related U.S. Application Data
(60) Provisional application No. 61/594,671, filed on Feb. 3, 2012.

(51) Int. Cl. C07K 16/18

(52) U.S. Cl. CPC C07K 16/18; C07K 2317/14
USPC 530/388.2; 435/69.6

(57) ABSTRACT

The disclosure provides a method of reducing aggregates in a preparation of monoclonal antibody by modifying at least three parameters in the bioreactor culture process.

### FIELD OF THE INVENTION

The present invention relates to the field of monoclonal antibody production.

### BACKGROUND

Monoclonal antibodies (mAbs) are an important class of biopharmaceuticals. They represent one of the best selling classes of biologics, with combined US sales reaching about $16.9 billion in 2009 (Aggarwal, 2010). MAbs are commonly used as diagnostic agents and as drugs, especially for treatment of various types of cancers and chronic inflammatory conditions. Currently, mAbs offer patients many new treatment options that are more effective, safer and more convenient than other traditional treatments (Jain & Kumar, 2008).

Antibodies to human delta-like antigen 4 (“DLL4”) are among those antibodies that have therapeutic applications that include treatment of various cancers. Several human monoclonal antibodies specific for DLL4 are described in U.S. patent application no. 2010/01963850.

MAbs for therapeutic applications can be expressed using Chinese Hamster Ovary (“CHO”) cells. Genes encoding such mAbs can be cloned and transfected into a CHO cell line which permits production of sufficient quantities of the mAb for clinical and commercial use. CHO cell clones transfected with the genes encoding the mAbs, upon expression and protein affinity (e.g., protein A) purification, may yield unacceptably high levels of antibody aggregate.

MAb aggregation is a major concern in therapeutic protein production. World Health Organization (WHO) standards limit the aggregate level in commercial intravenous immunoglobulin products to less than 5% (Pan et al., 2009). Considered a contaminant, aggregates reduce product purity and quality. They may cause an immunogenic response in patients (Barnard et al., 2010). In addition, aggregates may mechanically block capillaries causing reduced microcirculation in postischemic patients (Shellekens, 2005; Rosenberg, 2006). Accordingly, if aggregates cannot be reduced to an acceptable level below the WHO limits, an antibody with therapeutic potential may be dropped from development.

Aggregates may form at any step in the manufacturing process, including during culture of mammalian, e.g., CHO, cells. If aggregation for a mAb could be reduced at the cell culture stage, this would be highly beneficial as it would significantly reduce the downstream manufacturing burden and result in substantial overall process yield improvement.

Accordingly, a fermentation process for CHO cells expressing a DLL4 mAb that reduces formation of aggregates during cell culture is discussed herein. The fermentation process can yield a protein A-purified product from the culture having an aggregation level less than 5%.

### SUMMARY OF THE INVENTION

In accordance with the invention, an embodiment encompassed by the invention provides a method of producing an anti-DLL4 monoclonal antibody. Mammalian cells that express the monoclonal antibody are cultured at a temperature of about 36.5° C., a pH of about 6.85, and a starting osmolality of about 320 mOsm/kg H₂O. In another embodiment mammalian cells that express the monoclonal antibody are cultured at a temperature of about 37° C., a pH of about 7.0, and a starting osmolality of about 320 mOsm/kg H₂O. The expressed antibody is recovered from the culture supernatant.

Another embodiment encompassed by the invention is a method of reducing aggregates of an anti-DLL4 monoclonal antibody. A CHO cell that secretes the anti-DLL4 monoclonal antibody is cultured under conditions of temperature, pH, and osmolality that produce less aggregate than culture of the same mAb-producing CHO cell under conditions comprising a temperature of 36.5° C., a pH of 6.8, and a starting osmolality of 320 mOsm/kg H₂O in a bioreactor. The anti-DLL4 monoclonal antibody comprises a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO:3, a VL CDR1 comprising the amino acid sequence of SEQ ID NO:4, a VL CDR2 comprising the amino acid sequence of SEQ ID NO:5, and a VL CDR3 comprising the amino acid sequence of SEQ ID NO:6.

The method can comprise a multi-part feed. The multi-part feed can comprise a two-part feed. Alternatively the method can comprise a single feed. Glucose can be added to the culture to control the glucose level. Where glucose is added in the method, the addition of glucose is not considered a feed.

It is to be understood that both the foregoing general description and the following detailed description are exemplary and explanatory only and are not restrictive of the claims.

The accompanying drawings, which are incorporated in and constitute a part of this specification, illustrate several embodiments encompassed by the invention, and together with the description, serve to explain some of the principles of the invention.

### BRIEF DESCRIPTION OF THE DRAWINGS

FIG. 1 presents an HPLC profile showing antibody monomer and aggregate peaks.

FIG. 2 presents aggregate levels compared between twelve 1 L bioreactors with different cell culture conditions.

FIG. 3A-3F present a Design of Experiments (DoE) prediction profiler showing the relationship between temperature, osmolality, and pH as cell culture parameters in a linear model, and their impacts on titer and aggregate levels (post Protein A purification).

FIG. 4 presents the cell growth pattern observed in previous (red/bottom line) and new optimised (green/top line) fermentation process.

FIG. 5 presents a Design of Experiments Contour Profiler presenting the operating window for the cell culture process to achieve the aggregation level below 2% and titre above 6 g/L without reaching peak viable cell number higher than 24×10⁶ viable cells/mL.

FIG. 6 presents contour plots showing the relationship between temperature, osmolality, and pH as cell culture parameters in a quadratic model, and their impacts on titer and aggregate levels (post Protein A purification). Contour plots show the titre (top plots) and aggregate (bottom plots) range predictions as a function of pH (X axis), Temperature (Y axis) and osmolality (indicated above the plots).

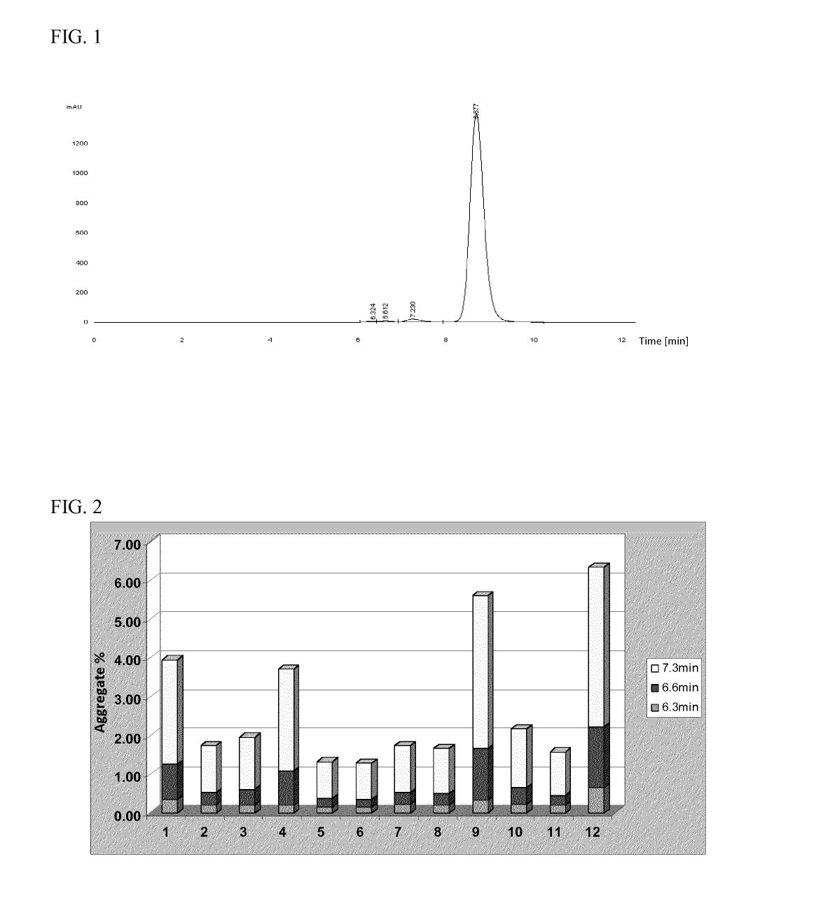

### DETAILED DESCRIPTION

Reference will now be made to various embodiments encompassed by the invention.

The disclosure provides a method of reducing aggregates of an anti-DLL4 monoclonal antibody (mAb) recovered from cell culture or from affinity-purified cell culture supernatant. The aggregates may also be reduced by culturing a first mammalian cell line that secretes the mAb under conditions of temperature, pH, and osmolality that produce less aggregate than culture of the same mammalian cell line that secretes the mAb under conditions including a temperature of 36.5° C., a pH of 6.8, and starting osmolality of 320 mOsm/kg H₂O in a bioreactor using a single feed. If desired, a second mammalian cell line that produces lower levels of mAb aggregate than the first mammalian cell line may be selected and substituted for the first mammalian cell line.

The pH, temperature, and starting osmolality culture conditions that reduce the aggregates of the anti-DLL4 mAb may be: pH 7.0, temperature 34° C., and starting osmolality 400 mOsm/kg H₂O; or pH 6.85, temperature 35.5° C., and starting osmolality 360 mOsm/kg H₂O; or pH 6.7, temperature 37° C., and starting osmolality 400 mOsm/kg H₂O; or pH 6.7, temperature 34° C., and starting osmolality 320 mOsm/kg H₂O; or pH 7.0, temperature 37° C., and starting osmolality 320 mOsm/kg H₂O; or pH 7.0, temperature 37° C., and starting osmolality 400 mOsm/kg H₂O; or pH 6.85, temperature 35.5° C., and starting osmolality 360 mOsm/kg H₂O; or pH 7.0, temperature 34° C., and starting osmolality 320 mOsm/kg H₂O; or pH 6.7, temperature 37° C., and starting osmolality 320 mOsm/kg H₂O; or pH 6.85, temperature 36.5° C., and starting osmolality 320 mOsm/kg H₂O.

The pH, temperature, and starting osmolality culture conditions that reduce the aggregates of the anti-DLL4 mAb to 5% or less may be a pH of between about 6.80 and about 7.00, osmolality of between about 320 and about 400 mOsm/kg H₂O, and a temperature between about 34.5° C. and about 36.5° C.

The anti-DLL4 mAb may be recovered from the cell culture following clarification and/or may be affinity purified utilizing, e.g., protein A affinity chromatography.

In some other embodiments of the various aspects disclosed herein, the culture temperature may be 37° C., or may be about 36.5° C. In one embodiment, the culture temperature is about 37.0° C. In another embodiment, the culture temperature is held at 37.0° C. within the margin of error of a bioreactor culture system. In yet another embodiment, the culture temperature is held at 37.0° C. within the margin of error of a DASGIP 1 L fed-batch bioreactor. In still other embodiments, the culture temperature may be 37.0° C.±0.1. In one embodiment, the culture temperature is 37° C.

In some other embodiments of the various aspects disclosed herein, the culture temperature may be 36.5° C., or may be about 36.5° C. In one embodiment, the culture temperature is about 36.5° C. In another embodiment, the culture temperature is held at 36.5° C. within the margin of error of a bioreactor culture system. In yet another embodiment, the culture temperature is held at 36.5° C. within the margin of error of a DASGIP 1 L fed-batch bioreactor. In still other embodiments, the culture temperature may be 36.5° C.±0.1. In one embodiment, the culture temperature is 36.5° C.

In some embodiments of the various aspects disclosed herein, the culture pH is about 7.0. In one embodiment, the culture pH is held at 7.0 within the margin of error of a bioreactor culture system. In another embodiment, the culture pH is held at 7.0±0.1 in a DASGIP 1 L fed-batch bioreactor. In one embodiment, the culture pH is 7.0. In some embodiments of the various aspects disclosed herein, the culture pH is about 6.85. In one embodiment, the culture pH is held at 6.85 within the margin of error of a bioreactor culture system. In another embodiment, the culture pH is held at 6.85±0.1 in a DASGIP 1 L fed-batch bioreactor. In one embodiment, the culture pH is 6.85. The pH may be adjusted during the culture process, for example, by adding alkali solution, sodium bicarbonate or CO₂ gas.

In some embodiments of the various aspects disclosed herein, the osmolality of the culture medium at the start of culture is about 320 mOsm/kg H₂O. In one embodiment, the osmolality of the culture medium at the start of culture is 320 mOsm/kg H₂O±1.0. In another embodiment, the osmolality of the culture medium at the start of culture is 320 mOsm/kg H₂O. In one embodiment, the culture medium is an animal protein-free medium. Osmolality of the medium can be adjusted, for example, by adding a salt such as NaCl. The culture medium may be any well known in the art or may be a media custom made by the user.

In some embodiments of the various aspects disclosed herein, the culture process may include a 2-part feed. In these embodiments, the feed is present as a two-part (i.e., stored in 2 separate containers) concentrate and each part of the concentrate is added individually to the culture. One of skill in the art of antibody production is able to identify commercially available feeds and may custom develop feeds for cell culture processes. Feeds may contain media or grouped media components such as amino acids, vitamins, iron, lipids, and trace elements. Feeds may be delivered to cells in one-part, or two-parts, or three-parts, or more parts. Commercially available feeds include IS CHO FEED CD™ (Irvine Scientific) and CHO CD EfficientFeed™ (Invitrogen).

In one embodiment of the various aspects disclosed herein, an animal protein-free medium with starting osmolality of 320 mOsm/kg H₂O is used to culture mAb-secreting cells in a bioreactor where the temperature is set to 37.0° C. and the pH is held at 7.0±0.1 during the culture, the culture process includes a 2-part feed.

In another embodiment of the various aspects disclosed herein, an animal protein-free medium with starting osmolality of 320 mOsm/kg H₂O is used to culture mAb-secreting cells in a bioreactor where the temperature is set to 36.50° C. and the pH is held at 6.85±0.1 during the culture, the culture process includes a 2-part feed.

In one embodiment of the various aspects disclosed herein, the method reduces the percentage of aggregates of the anti-DLL4 mAb in the supernatant of cultured cells expressing the anti-DLL4 mAb and the percentage anti-DLL4 mAb aggregate reduction may be measured in a sample of the cell culture supernatant, or in in a sample of the cell culture supernatant following clarification, or in a sample of the cell culture supernatant following clarification and affinity purification. In another embodiment, the reduction of the percentage of aggregates of the anti-DLL4 antibody may be a reduction of aggregates recovered in supernatant from the cells following clarification and the percentage aggregates may be determined from a sample of the clarified cell culture supernatant or may be determined from a sample of the clarified cell culture supernatant following affinity purification. The reduction of aggregates of the anti-DLL4 antibody may be a reduction of aggregates recovered from affinity-purified cell culture supernatant of the cells expressing the DLL4 mAb. The percentage aggregates may be determined from a sample of the affinity-purified cell culture supernatant.

If the supernatant of the cells expressing the mAb is clarified, then larger particles, e.g., cells debris or cells, are removed from the harvested cell culture supernatant. Methods of clarifying cell culture supernatants are known to those of skill in the art and include flow filtration, depth filtration, centrifugation, and centrifugation followed by one or more filtration steps.

The supernatant of the cells expressing the mAb or clarified supernatant of the cells expressing the mAb may be affinity purified. One of skill in the art is well aware of various affinity purification methods that may be used to purify antibodies. These include, without limitation, purification by Protein A, Protein G, Protein A/G, or Protein L affinity chromatography. Those of skill in the art are also aware that affinity chromatography methods include those which employ immobilized antigen, i.e., DLL4, to which the mAb specifically binds.

Another aspect of the disclosure provides a method of reducing aggregate content in a protein A-purified mAb product to less than about 5%. The method comprises steps of: culturing a mammalian cell line that expresses an anti-DLL4 mAb in a culture medium having a starting osmolality of about 320 mOsm/kg H₂O, at a temperature of about 37° C., and at a pH of about 7.0. Another aspect of the disclosure provides a method of reducing aggregate content in a protein A-purified mAb product to less than about 5%. The method comprises steps of: culturing a mammalian cell line that expresses an anti-DLL4 mAb in a culture medium having a starting osmolality of about 320 mOsm/kg H₂O, at a temperature of about 36.5° C., and at a pH of about 6.85. The mammalian cell line expresses an anti-DLL4 antibody comprising a heavy chain variable domain comprising CDR1, CDR2, and CDR3 amino acid sequences as set forth in SEQ ID NO:1, SEQ ID NO: 2, and SEQ ID NO: 3, respectively, and a light chain variable domain comprising CDR1, CDR2, and CDR3 amino acid sequences as set forth in SEQ ID NO:4, SEQ ID NO: 5, and SEQ ID NO: 6, respectively. The culturing process comprises utilizing a two part feed to feed the cells during the culturing to thereby reduce aggregate formation in the culture supernatant. The antibody expressed by the mammalian cell line is then recovered from the culture supernatant. The antibody may be purified using an affinity chromatography step, e.g., protein A.

In some embodiments, the aggregate content is measured by HPLC-SEC. In one embodiment, the aggregate content is less than about 5%, about 4%, about 3%, or about 2%. In one embodiment, the aggregate content is about 1.2%, about 1.3%, about 1.4%, about 1.5%, about 1.6%, about 1.7%, about 1.8%, or about 1.9%.

The disclosure also provides a method of producing an anti-DLL4 monoclonal antibody comprising steps of: culturing a Chinese Hamster Ovary (CHO) cell that expresses an antibody heavy chain variable domain as set forth in SEQ ID NO: 7 and a light chain variable domain as set forth in SEQ ID NO: 8 in a culture medium with a starting osmolality of about 320 mOsm/kg H₂O, at a temperature of about 37° C., and a pH of about 7; using a two part feed to feed the cells during the culturing process; and recovering the expressed anti-DLL4 antibody from the culture supernatant.

The disclosure also provides a method of producing an anti-DLL4 monoclonal antibody comprising steps of: culturing a Chinese Hamster Ovary (CHO) cell that expresses an antibody heavy chain variable domain as set forth in SEQ ID NO: 7 and a light chain variable domain as set forth in SEQ ID NO: 8 in a culture medium with a starting osmolality of about 320 mOsm/kg H₂O, at a temperature of about 36.5° C., and a pH of about 6.85; using a two part feed to feed the cells during the culturing process; and recovering the expressed anti-DLL4 antibody from the culture supernatant.

The expressed antibody may be recovered from the culture supernatant by protein A chromatography.

The disclosure also provides for a method of producing an anti-DLL4 monoclonal antibody, comprising:
(a) culturing a mammalian cell line that expresses the anti-DLL4 mAb in a culture medium at about 37° C. and about pH 7.0,
   wherein the anti-DLL4 antibody comprises a heavy chain variable domain comprising CDR1, CDR2, and CDR3 amino acid sequences as set forth in SEQ ID NO:1, SEQ ID NO: 2, and SEQ ID NO: 3, respectively, and a light chain variable domain comprising CDR1, CDR2, and CDR3 amino acid sequences as set forth in SEQ ID NO:4, SEQ ID NO: 5, and SEQ ID NO: 6, respectively; and
   wherein the culture medium has a starting osmolality of about 320 mOsm/kg H₂O;
(b) using a two-part feed to feed the cells during the culturing process; and
(c) recovering the expressed antibody from the culture supernatant.

In another embodiment the disclosure also provides for a method of producing an anti-DLL4 monoclonal antibody, comprising:
(a) culturing a mammalian cell line that expresses the anti-DLL4 mAb in a culture medium at about 36.5° C. and about pH 6.85,
   wherein the anti-DLL4 antibody comprises a heavy chain variable domain comprising CDR1, CDR2, and CDR3 amino acid sequences as set forth in SEQ ID NO:1, SEQ ID NO: 2, and SEQ ID NO: 3, respectively, and a light chain variable domain comprising CDR1, CDR2, and CDR3 amino acid sequences as set forth in SEQ ID NO:4, SEQ ID NO: 5, and SEQ ID NO: 6, respectively; and
   wherein the culture medium has a starting osmolality of about 320 mOsm/kg H₂O;
(b) using a two-part feed to feed the cells during the culturing process; and
(c) recovering the expressed antibody from the culture supernatant.

The disclosure also provides a method of producing an anti-DLL4 monoclonal antibody comprising: culturing a Chinese Hamster Ovary (CHO) cell that expresses the antibody heavy and light chains in a culture medium with a starting osmolality of about 320 mOsm/kg H₂O, at a temperature of about 37° C. and a pH of about 7.0, wherein the CHO cell is fed during the culturing process using a 2 part feed; and recovering the antibody from the culture supernatant.

The disclosure also provides a method of producing an anti-DLL4 monoclonal antibody comprising: culturing a Chinese Hamster Ovary (CHO) cell that expresses the antibody heavy and light chains in a culture medium with a starting osmolality of about 320 mOsm/kg H₂O, at a temperature of about 36.5° C. and a pH of about 6.85, wherein the CHO cell is fed during the culturing process using a 2 part feed; and recovering the antibody from the culture supernatant.

In any aspect of producing a mAb by recovering the mAb from a culture supernatant as provided herein, the recovery step may comprise purifying the mAb on protein A. The protein A-purified mAb may have an aggregate content of less than about 5%, about 4%, about 3%, or about 2%. In one embodiment, the protein A-purified mAb has an aggregate content of about 1.2%, about 1.3%, about 1.4%, about 1.5%, about 1.6%, about 1.7%, about 1.8%, or about 1.9%.

In any of the aspects or embodiments described herein, the mammalian cell line that expresses an antibody may be chosen from Chinese hamster ovary (CHO) cells, NS0 cells, or PER.C6 (ECACC no. 96022940) cells. In one embodiment, the mammalian cell line is CHO. The CHO cell line may be CHOK1SV cells (Lonza).

The cell line that secretes a mAb may be transfected with an appropriate gene or genes that express the mAb.

In any of the aspects or embodiments described herein, if a percentage of aggregate or a percentage of monomer in a mAb product is to be determined, these percentages may be determined utilizing techniques such as field-flow fractionation, analytical ultracentrifugation, dynamic light scattering, size exclusion chromatography, or other methods known in the art. For example, percentage may be determined employing HPLC-SEC analysis of protein A purified mAb samples to quantitate amounts of monomer and aggregates in the total mAb amount.

The disclosure also provides for an anti-DLL4 mAb produced according to any of the described culture methods. The disclosure further provides for a composition comprising the DLL4 mAb produced according to of the methods.

The anti-DLL4 mAb product or composition may comprise less than about 1.4% aggregate following protein A purification. The percentage of aggregate may be determined by HPLC-SEC.

The disclosure also provides for a pharmaceutical composition comprising, consisting essentially of, or consisting of an anti-DLL4 mAb produced by a method as described herein and a pharmaceutically acceptable carrier.

In any of the various aspects or embodiments, the anti-DLL4 mAb may be a mAb that is a human IgG1 mAb that binds DLL4. The mAb may be an anti-DLL4 antibody that comprises a variable heavy chain amino acid sequence comprising CDR1, CDR2, and CDR3 amino acid sequences as set forth in SEQ ID NO:1, SEQ ID NO: 2, and SEQ ID NO: 3, respectively, and a variable light chain amino acid sequence comprising CDR1, CDR2, and CDR3 amino acid sequences as set forth in SEQ ID NO: 4, SEQ ID NO: 5, and SEQ ID NO: 6, respectively.

In other aspects or embodiments, the mAb may be an anti-DLL4 antibody that comprises a heavy chain polypeptide comprising the sequence of SEQ ID NO: 7. The mAb may be an anti-DLL4 antibody that comprises a light chain polypeptide comprising the sequence of SEQ ID NO:8. The mAb may be an anti-DLL4 antibody that comprises a heavy chain polypeptide comprising the sequence of SEQ ID NO:7 and a light chain polypeptide comprising the sequence of SEQ ID NO:8. The anti-DLL4 mAb may be a fully human monoclonal antibody.

In still other aspects or embodiments, the mAb is an anti-DLL4 antibody that comprises a variable heavy chain amino acid sequence comprising at least one, at least two, or at least three of the CDRs of the antibody encoded by the polynucleotide in the plasmid designated Mab21H3VH, which was deposited at the American Type Culture Collection (ATCC) under number PTA-9501 on Sep. 17, 2008.

In yet other aspects or embodiments, the mAb is an anti-DLL4 antibody that comprises a variable light chain amino acid sequence comprising at least one, at least two, or at least three of the CDRs of the antibody encoded by the polynucleotide in the plasmid designated Mab21H3VLOP, which was deposited at the ATCC under number PTA-9500 on Sep. 17, 2008.

In other aspects or embodiments, the mAb is an anti-DLL4 antibody that comprises a variable heavy chain amino acid sequence comprising at least one, at least two, or at least three of the CDRs of the antibody encoded by the polynucleotide in the plasmid designated Mab21H3VH, which was deposited at the ATCC under number PTA-9501 on Sep. 17, 2008; and comprises a variable light chain amino acid sequence comprising at least one, at least two, or at least three of the CDRs of the antibody encoded by the polynucleotide in the plasmid designated Mab21H3VLOP, which was deposited at the ATCC under number PTA-9500 on Sep. 17, 2008. The mAb may be an anti-DLL4 antibody that comprises all three heavy chain CDR amino acid sequences encoded by the polynucleotide in the plasmid designated Mab21H3VH, which was deposited at the ATCC under number PTA-9501 on Sep. 17, 2008, and all three light chain CDR amino acid sequences encoded by the polynucleotide in the plasmid designated Mab21VLOP, which as deposited at the ATCC under number PTA-9500 on Sep. 17, 2008. The mAb may be an anti-DLL4 antibody that comprises the heavy chain amino acid sequence encoded by the polynucleotide in the plasmid designated Mab21H3VH, which was deposited at the ATCC under number PTA-9501 on Sep. 17, 2008, and the light chain amino acid sequence encoded by the polynucleotide in the plasmid designated Mab21VLOP, which as deposited at the ATCC under number PTA-9500 on Sep. 17, 2008.

The various methods of reducing aggregates of an anti-DLL4 monoclonal antibody (mAb) recovered from cell culture or from affinity-purified cell culture supernatant may further result in increased mAb titer. The methods may increase mAb titre by at least 10%, by at least 20%, by at least 25%, by at least 30%, by at least 40%, by at least 50%, by at least 60%, by at least 70%, by at least 75%, by at least 80%, by at least 90%, by at least 100%, by at least 125%, by at least 150%, by at least 175%, or by at least 200%. The mAb titer may be at least 1 g/L, at least 2 g/L, at least 3 g/L, at least 4 g/L, at least at least 5 g/L, at least 5.5 g/L, at least 6 g/L, at least 6.25 g/L, at least 6.5 g/L, at least 6.75 g/L, at least 7 g/L, at least 7.25 g/L, at least 7.5 g/L, at least 7.75 g/L, at least 8 g/L or at least 8.5 g/L.

It is noted that those of ordinary skill in the art can readily accomplish CDR determinations. See for example, Kabat et al., Sequences of Proteins of Immunological Interest, Fifth Edition, NIH Publication 91-3242, Bethesda Md. (1991), vols. 1-3. Kabat provides multiple sequence alignments of immunoglobulin chains from numerous species antibody isotypes. The aligned sequences are numbered according to a single numbering system, the Kabat numbering system. The Kabat sequences have been updated since the 1991 publication and are available as an electronic sequence database (latest downloadable version 1997). Any immunoglobulin sequence can be numbered according to Kabat by performing an alignment with the Kabat reference sequence. Accordingly, the Kabat numbering system provides a uniform system for numbering immunoglobulin chains.

Further embodiments, features, and the like regarding a process for reducing aggregates and the product produced thereby as disclosed herein are provided in additional detail below.

### Terminology

Unless otherwise defined, scientific and technical terms used herein shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art.

Standard techniques are typically used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (3rd ed., Cold Spring Harbor Laboratory Press, Cold Spring Harbor, N.Y. (2001)), which is incorporated herein by reference. The nomenclatures utilized in connection with, and the laboratory procedures and techniques of, analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, and delivery, and treatment of patients.

As utilized in accordance with the present disclosure, the following terms, unless otherwise indicated, shall be understood to have the following meanings:

The term “and/or” as used herein is to be taken as specific disclosure of each of the two specified features or components with or without the other. For example “A and/or B” is to be taken as specific disclosure of each of (i) A, (ii) B and (iii) A and B, just as if each is set out individually.

The term “about” as used herein in connection with any and all values (including lower and upper ends of numerical ranges) means any value having an acceptable range of deviation of up to ±10% (and values there between, e.g., ±0.5%, ±1%, ±1.5%, ±2%, ±2.5%, ±3%, ±3.5%, ±4%, ±4.5%, ±5%, ±5.5%, ±6%, ±6.5%, ±7%, ±7.5%, ±8%, ±8.5%, ±9%, ±9.5%).

As used herein and in the appended claims, the singular forms “a,” “an,” “or,” and “the” include plural referents unless the context clearly dictates otherwise.

The term “antibody” or “antibodies” refers to a polypeptide or group of polypeptides that are comprised of at least one binding domain that is formed from the folding of polypeptide chains having three-dimensional binding spaces with internal surface shapes and charge distributions complementary to the features of an antigenic determinant of an antigen chain. Native antibodies are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Light chains are classified as either lambda chains or kappa chains based on the amino acid sequence of the light chain constant region. The variable domain of a kappa light chain may also be denoted herein as VK. The term “variable region” may also be used to describe the variable domain of a heavy chain or light chain. Particular amino acid residues are believed to form an interface between the light and heavy chain variable domains. The variable regions of each light/heavy chain pair form an antibody binding site. Such antibodies may be derived from any mammal, including, but not limited to, humans, monkeys, pigs, horses, rabbits, dogs, cats, mice, etc.

Antibodies include immunoglobulin molecules, i.e., molecules that contain an antigen-binding site. Immunoglobulin molecules can be of any type (e.g., IgG, IgE, IgM, IgD, IgA and IgY), class (e.g., IgG1, IgG2, IgG3, IgG4, IgA1 and IgA2) or subclass.

A “human antibody” is an antibody derived from a human or an antibody obtained from a transgenic organism that has been engineered to produce human antibodies in response to antigenic challenge. A human antibody may also include an antibody wherein the heavy and light chains are encoded by a nucleotide sequence derived from one or more sources of human DNA. A fully human antibody can be constructed by genetic or chromosomal transfection methods, phage display technology (e.g., U.S. Pat. No. 5,969,108), or in vitro activated B cells (e.g., U.S. Pat. Nos. 5,567,610 and 5,229,275). An antibody may be from any species.

The term “mAb” refers to a monoclonal antibody. A monoclonal antibody is an antibody derived from a single cellular source, such as a hybridoma, a transformed cell, or a cell made to express the genes encoding an antibody by transfection or other technique.

Examples of suitable mammalian cell lines include Chinese Hamster Ovary (“CHO”), NS0, or PER.C6 (ECACC no. 96022940) cell lines. Any of these mammalian cell lines can be generally transfected with one or more recombinant vectors that encode the heavy and light chains, or fragments thereof, of a mAb of interest. The transfected cells secrete a mAb comprising the encoded heavy and light chains into the cell culture medium (supernatant).

Examples of mAbs suitable for use in the methods and compositions of the disclosure are the human anti-DLL4 antibodies described in US2010/0196385 or WO 2010/032060, each of which are incorporated by reference. The amino acid sequence of the variable region of the heavy chain and light chain of an example of one such anti-DLL4 mAb is set forth in SEQ ID NO: 30 and SEQ ID NO: 50, respectively, of US2010/0196385. Other examples of monoclonal antibodies suitable for use in the methods and compositions provided herein include anti-DLL4 antibodies that comprise a heavy chain variable domain comprising CDR1: NYGIT (SEQ ID NO:1); CDR2: WISAYNGNTNYAQKLQD (SEQ ID NO:2); and CDR3: DRVPRIPVTTEAFDI (SEQ ID NO: 3), and a light chain variable domain comprising CDR1: SGSSSNIGSYFVY (SEQ ID NO:4); CDR2: RNNQRPS (SEQ ID NO;5); and CDR3: AAWDDSLSGHWV (SEQ ID NO: 6). In one embodiment, an anti-DLL4 mAb suitable for use in the methods and compositions provided herein comprises a heavy chain variable domain as set forth in SEQ ID NO: 7 and a light chain variable domain as set forth in SEQ ID NO: 8.

SEQ ID NO: 7:
Gln Val Gln Leu Val Gln Ser Gly Ala
Glu Val Lys Lys Pro Gly Ala Ser Val
Lys Val Ser Cys Lys Ala Ser Gly Tyr
Thr Phe Thr Asn Tyr Gly Ile Thr Trp
Val Arg Gln Ala Pro Gly Gln Gly Pro
Glu Trp Met Gly Trp Ile Ser Ala Tyr
Asn Gly Asn Thr Asn Tyr Ala Gln Lys
Leu Gln Asp Arg Val Thr Val Thr Thr
Asp Thr Ser Thr Ser Thr Ala Tyr Met
Glu Leu Arg Ser Leu Arg Ser Asp Asp
Thr Ala Val Tyr Tyr Cys Ala Arg Asp
Arg Val Pro Arg Ile Pro Val Thr Thr
Glu Ala Phe Asp Ile Trp Gly Gln Gly
Thr Met Val Thr Val Ser Ser
SEQ ID NO: 8:
Gln Ser Val Leu Thr Gln Pro Pro Ser
Ala Ser Gly Thr Pro Gly Gln Arg Val
Thr Ile Ser Cys Ser Gly Ser Ser Ser
Asn Ile Gly Ser Tyr Phe Val Tyr Trp
Tyr Gln Gln Leu Pro Gly Thr Ala Pro
Lys Leu Leu Ile Tyr Arg Asn Asn Gln
Arg Pro Ser Gly Val Pro Asp Arg Phe
Ser Gly Ser Glu Ser Gly Thr Ser Ala
Ser Leu Ala Ile Ser Gly Leu Arg Ser
Glu Asp Glu Ala Asp Tyr Tyr Cys Ala
Ala Trp Asp Asp Ser Leu Ser Gly His
Trp Val Phe Gly Gly Gly Thr Lys Leu
Thr Val Leu

The term “DLL4” refers to the molecule that is DLL4 protein, also known as Delta-like protein 4 precursor, Drosophila Delta homolog 4, hdelta2, MGC126344, or UNQ1895/P R04341.

The term “binding fragment(s)” includes single-chain Fvs (scFv), single-chain antibodies, single domain antibodies, domain antibodies, Fv fragments, Fab fragments, F(ab′) fragments, F(ab′)₂ fragments, antibody fragments that exhibit the desired biological activity, disulfide-stabilised variable region (dsFv), dimeric variable region (Diabody), anti-idiotypic (anti-Id) antibodies, intrabodies, linear antibodies, single-chain antibody molecules and multispecific antibodies formed from antibody fragments and epitope-binding fragments of any of the above. “Binding fragments” of an antibody are produced by recombinant DNA techniques, or by enzymatic or chemical cleavage of intact antibodies. Those of skill in the art are well aware of examples of “binding fragments(s)” as would be useful in the methods and compositions provided herein.

Antibodies, as described herein, can be prepared in a mixture with a pharmaceutically acceptable carrier.

Embodiments of the invention include sterile pharmaceutical formulations of antibodies. Sterile formulations can be created, for example, by filtration through sterile filtration membranes, prior to or following lyophilization and reconstitution of the antibody. Antibodies ordinarily will be stored in lyophilized form or in solution. Therapeutic antibody compositions generally are placed into a container having a sterile access port, for example, an intravenous solution bag or vial having an adapter that allows retrieval of the formulation, such as a stopper pierceable by a hypodermic injection needle.

A “therapeutically effective” amount is an amount that provides some improvement or benefit to the subject. Stated in another way, a “therapeutically effective” amount is an amount that provides some alleviation, mitigation, and/or decrease in at least one clinical symptom. Further, those skilled in the art will appreciate that the therapeutic effects need not be complete or curative, as long as some benefit is provided to the subject.

### EXAMPLES

The following examples, including the experiments conducted and results achieved, are provided for illustrative purposes only and are not to be construed as limiting upon the teachings herein.

Cell lines producing a mAb of interest are routinely cloned and even subcloned by selecting individual cells from a culture for further expansion and analysis. Although the mAb produced remains the same among the clones (e.g., it has the same amino acid sequence), both the level of mAb produced (titer) and the level of aggregates formed may vary among the clones. It is therefore standard practice to prepare a number of clones, determine their mAb production characteristics, and then select a single clone for large scale development. One or a handful of other clones may be selected as back-ups. This selection process is both time consuming and costly.

When CHO subclones of a human anti-DLL4 mAb were prepared, some of the clones produced a high percentage of aggregate relative to monomer compared to other clones producing transfected with the same DNA sequence.

TABLE 1
Percentage of monomer produced by exemplary clones.

| Clone | Monomer %* |
|---|---|
| 10-516 | 97.7 |
| 10-520 | 96.8 |
| 21-62 | 98.3 |
| 22-93 | 98.5 |
| 31-121 | 91.1 |
| 7-282 | 92.9 |

*Post Protein A purification (HPLC-SEC).

As shown in Table 1, clone 31-121 produced particularly high levels of aggregate. Like the other clones, it comprises a heavy chain variable domain as set forth in SEQ ID NO: 7 and a light chain variable domain as set forth in SEQ ID NO: 8. It has an isometric point of 9.0 and as a monomer is 148 kDa.

If a culture process could be developed for clone 31-121 that would reduce aggregate levels, it would become a potentially useful clone for commercial development. In addition, culture conditions that reduced aggregate levels for clone 31-121 could reasonably be expected to reduce the levels of aggregate produced using other clones expressing the antibody.

### Example 1

### Relationship Between Cell Culture Parameters and the Level and Composition of Aggregates after Protein A Purification

A method was developed that lowered levels of aggregation in the fermentation process without decreasing the mAb productivity profile. Production of higher monomer purity mAbs at fermentation is beneficial for downstream purification processes and would result in improvement of the final process yields.

### Cell Culture

Cell Culture Maintenance:

Clone 31-121 cells were maintained in 250 mL shake flasks containing 50 mL of animal protein free medium (M18). Cells were seeded at a cell seeding of 3×10⁵ viable cells/mL in in-house animal protein-free medium (M18) supplemented with glutamine synthetase inhibitor L-methionine sulfoximine (MSX). Cell cultures were maintained under continuous shaking at 140 rpm in an atmosphere of 5% of CO2/95% air at 36.5° C. and passaged every three days.

Cell Culture Processes in 1 L Bioreactors:

Cell culture processes were carried out in two blocks of six 1 L fed-batch bioreactors (DASGIP AG, Jülich, Germany). The cells were seeded at a cell density between 8 and 10×10⁵ viable cells/mL in M18 medium without any supplements. Cell cultures were maintained under continuous stirring of 150 rpm and then 175 rpm from day 7. Dissolved oxygen, pH and temperature were measured online using appropriate probes. This information was used to activate oxygen sparging to maintain dissolved oxygen, activate CO₂ sparging or pumping of alkali to maintain pH (with a deadband of 0.1 pH unit) and activate a heating blanket to maintain temperature. In addition, samples were taken for off-line measurement of cell counts ((Vi-Cell, Beckman Coulter, Inc., Fullerton, Calif., USA), pH, gases and nutrient analysis (BioProfile FLEX Analyzer, NOVA biomedical, Waltham, Mass., USA). Bioreactors were supplemented with glucose daily if the glucose level dropped below 4 g/L. In addition to glucose supplementation, the cultures were fed with two different types of feed (single part feed (M20a) or 2 part feed) depending on the bioreactor run and condition tested.

### Protein A Purification

Cell culture supernatants of 14 day cultures were clarified by centrifugation and filteriation through 1.2 μm, 0.45 μm and 0.2 μm pore-size membrane filters. The clarified cultures were subjected to Protein A affinity columns.

The protein A purification was performed using MabSelect SuRe (GE Healthcare, Uppsala, Sweden), packed into a Vantage-L 11 column (Millipore, Mass., USA). The MabSelect SuRe column was equilibrated with phosphate buffered saline and was then loaded with clarified cell culture supernatant, to a capacity of 30 g mAb per liter of resin. The column was then subjected to a requilibration and two wash steps before being eluted at low pH. All Protein A purification runs were performed using an AKTA avant controlled using Unicorn 6 software (both from GE Healthcare, Uppsala, Sweden).

The eluate from the Protein A purification was subjected to a low pH viral inactivation step. Eluates were titrated down using acetic acid, before being neutralized to pH 5.0 using untitrated Tris solution. The neutralized eluates were then filtered using a 0.2 μm filter, before being stored.

### SEC-HPLC Analysis of Aggregates in the Samples

Size Exclusion Chromatography (SEC) is an industry standard technique for detection and quantification of pharmaceutical protein aggregates (Gabrielson et al., 2005; Liu et al., 2009; Mahler et al., 2008). SEC was performed using a TSKgel 3000 SW_{XL} column (7.8 mm×30.0 cm; TOSOH Bioscience, Stuttgart, Germany) and a SW_{XL} guard column (6.0 mm×4.0 cm; TOSOH Bioscience, Stuttgart, Germany) connected to a Agilent 1100 equipped with a UV280 detector. Elution was performed using 0.1 M sodium phosphate and 0.1 M sodium sulphate buffer at pH 6.8 at flow rate of 1.0 mL/min for chromatographic separation on a HPLC system. Prior to use, the column was calibrated using BioRad gel filtration standard from Bio-Rad laboratories (Hercules, Calif., USA).

Samples were compared to each other by measuring area underneath the curve, for the peak of interest (mAb monomer peak or aggregate peaks):

$$Peak area = peak height [ mAU ] peak width [ s ] .$$

The percentage of aggregates in the samples was calculated by dividing the total aggregate peak areas by the total IgGs (monomer and aggregate) peak area:

$$Aggregate \% = \frac{Aggregate peak areas}{Aggregate peak areas + Monomer peak area} \times 100.$$

### DoE Analysis of Impact of Culture Parameters on Aggregate Formation and mAb Titer

Initial tests were performed in shake flasks with parameters such as feed type, initial media osmolality and seeding cell density screened for their impact on aggregate formation (data not shown). Other factors, such as temperature, pH and agitation rate, required a bioreactor system which is capable of tight control of these parameters. Based on obtained results, temperature, pH and starting osmolality of media were the major process cell culture factors having a significant impact on aggregate formation. During the shake flask experiments, the type of feed used was not seen to play an important role in terms of affecting mAb aggregate formation. However, use of the 2-part feed was seen to provide a beneficial effect in terms of mAb titre. As a result, the 2-part feed was used for all further experiments.

The aggregate level results were then analyzed by the Design of Experiments (“DoE”) JMP statistical tool (SAS, Cary, N.C., USA). DoE has proven very effective in distinguishing major and minor contributing factors. DoE is also known as one of the most economical and most accurate methods for performing process optimization. It not only provides statistical validation that a variable impacts the process, but it also accelerates understanding of the interrelationships among process variables, and indicates an optimum based on the interaction of the variables and their criticality to the process.

Three variable factors were included in the DoE full factorial design: temperature, pH, and starting osmolality of medium. The media osmolality was increased by adding NaCl. The ranges for each of the variables are shown in Table 2. Based on these input variables, an experimental matrix was generated (using JMP software from SAS, Cary, N.C., USA) consisting of 12 runs (8 corner points, 2 centre points and 2 reactors comparing single feed and two-part feed). Table 3 provides bioreactor conditions for each run. The experiments were carried out in 2 blocks of six bioreactors.

TABLE 2
Inducing factors ranges.

| Inducing Factor | Range |
|---|---|
| Temperature | 34-37° C. |
| pH | 6.7-7.0 |
| Osmolality | 320-400 mOsm/kg H₂O |

TABLE 3
Bioreactor conditions and feed

| | Cell culture conditions | | | |
|---|---|---|---|---|
| | | Temperature | Osmolality | |
| Bioreactor | pH | [° C.] | [mOsm/kg] | Feed type |
| 1 | 7.00 | 34 | 400 | 2 part feed |
| 2 | 6.85 | 35.5 | 360 | 2 part feed |
| 3 | 6.70 | 37 | 400 | 2 part feed |
| 4 | 6.70 | 34 | 320 | 2 part feed |
| 5 | 7.00 | 37 | 320 | 2 part feed |
| 6 | 7.00 | 37 | 400 | 2 part feed |
| 7 | 6.85 | 35.5 | 360 | 2 part feed |
| 8 | 7.00 | 34 | 320 | 2 part feed |
| 9 | 6.70 | 34 | 400 | 2 part feed |
| 10 | 6.70 | 37 | 320 | 2 part feed |
| 11 | 6.80 | 36.5 | 320 | 2 part feed |
| 12 | 6.80 | 36.5 | 320 | Single part |
| | | | | feed (M20a) |

All 12 fermenters were harvested on day 14 and clarified to remove cells and large particulates. The antibody product from each of the 12 bioreactors was then purified using Protein A chromatography. The aggregate content of the Protein A eluates were compared across the 12 experimental runs (FIG. 2).

The percentage composition of the three aggregate peaks (with retention times on SEC-HPLC of 6.3 min, 6.6 min and 7.2 min) was calculated from the total IgGs (monomer and aggregate peaks) produced. Interesting variations in the aggregate levels were observed ranging from slightly above 1% to more than 6%.

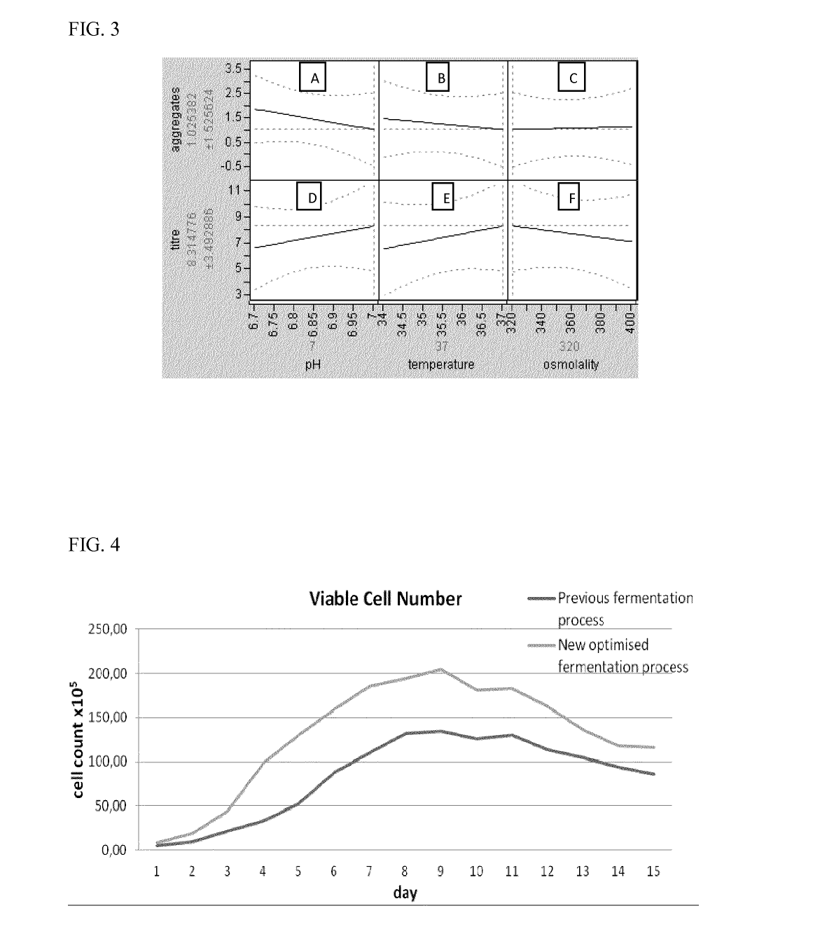

The results of aggregate levels obtained after bioreactor runs with different cell culture conditions were analysed using a DoE based software statistical tool (JMP SAS, Cary N.C., USA). The DoE software was used to generate a linear mathematical model describing the relationship between the tested factors and mAb aggregation as well as final mAb titre. FIG. 3A-F present the DoE-generated prediction profiler showing the relationship between the tested factors temperature (panels A, D), pH, (panels B, E) and osmolality (panels C, F) within the chosen ranges on both aggregation (panels A, B, C) and final mAb titer (panels D, E, and F). The profiler showed that when the pH and temperature levels are increasing, the aggregation is decreasing (panels A and B) but the mAb titer is increasing (panels D and E). There was no significant correlation observed between the starting osmolality of M18 cell culture medium and the aggregation level (panel C). However, the lower osmolality was beneficial for the final mAb titer (panel F).

The DoE prediction profiler indicates that to achieve less aggregation the cells should be cultured in medium with low osmolality and high temperature and pH. Within the chosen range of tested factors, the DoE prediction profiler indicates cells should be cultured in medium with starting osmolality of 320 mOsm/kg H₂O and cultured at pH 7.0 and 37° C. These conditions were predicted to lower mAb aggregation to only 1.03% and increase final mAb concentration to 8.3 g/L.

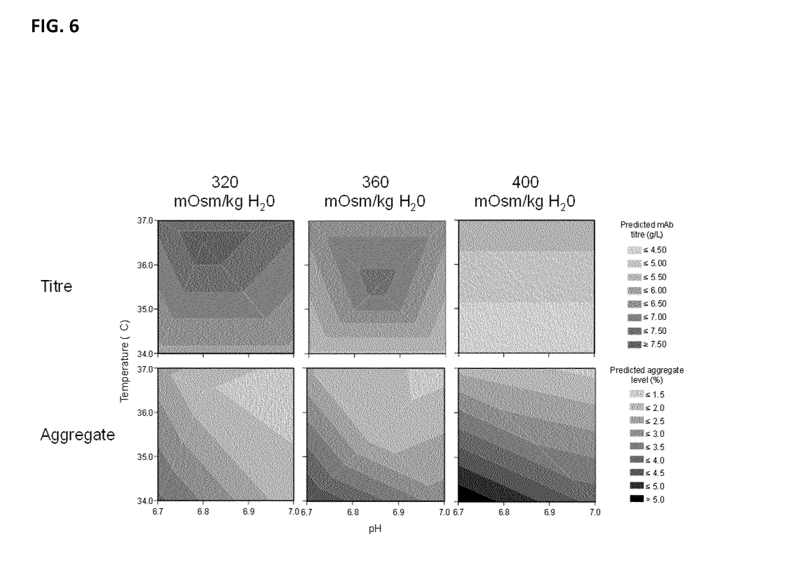

The results of aggregate levels obtained after bioreactor runs with different cell culture conditions were further analysed using the DoE based software statistical tool (JMP SAS, Cary N.C., USA) to generate a quadratic model describing the relationship between the tested factors and mAb aggregation as well as final mAb titre. FIG. 6 presents the DoE-generated contour plots showing the relationship between the tested factors temperature, pH, and osmolality within the chosen ranges on both aggregation and final mAb titer. The contour plots showed that the pH, temperature and osmolality levels can be optimized to decrease aggregation and increase mAb titer.

Within the chosen range of tested factors, the DoE contour plots indicate cells should be cultured in medium with starting osmolality of 320 mOsm/kg H₂O and cultured at pH 6.85 and temperature of 36.5° C.

Based on published data, it was expected that mAb aggregation would be reduced in cultures maintained at 34° C. when compared to those at 37° C. An increase in culture temperature should accelerate chemical reactions such as oxidation or deamidation of biopharmaceutical proteins. It may also cause temperature-induced unfolding of immunoglobulins which is another factor which can promote protein aggregation (Mahler et al., 2008; Brange et al., 1992). Lower culture temperatures should also slow down the cell growth (which was observed in the experiments performed for this study) as well as give more time for correct protein folding. However, the data generated from this study showed the opposite effect, with lower levels of mAb aggregation occurring in cultures maintained at higher temperatures. This would indicate that temperature is affecting the way in which the cells grow and thus some intracellular mechanism of mAb aggregation.

Cell culture medium pH may influence the electrostatic interactions between mAb molecules by affecting the charge distribution on the protein surfaces. By lowering the system pH, moving further from the isoelectric point of the mAb, it could be expected that increased charge densities would result in increased levels of like-charge repulsion between molecules. This in turn could be expected to reduce mAb aggregation. The results of the experiments performed in this study however showed that increasing the medium pH to 6.85 or 7.0, actually resulted in a reduction in mAb aggregation levels. Further reductions in mAb aggregation levels were observed when the cell culture medium pH was increased to a value of 7.2 (data not shown). The observations may be explained by the fact that under acidic conditions, protein cleavages are favoured (Idicula-Thomas & Balaji, 2007). Therefore, by increasing the cell culture medium pH from very weakly acidic (pH 6.7) to neutral and slightly alkali conditions (pH 7.2) these chemical reactions which may lead to protein aggregation, were prevented.

### Example 2

### Comparison Between New Fermentation Process and Previous Process which Generated High Aggregate Level

Cells were cultured in a bioreactor under the optimised conditions, as predicted by the results of the DoE based study. The performance of this new process was compared against that of the previous base case process which had generated high levels of aggregate with this particular clone. In addition to aggregate composition and final mAb titre, the peak viable cell number (VCN) reached during the process, the amount of alkali and glucose solutions dosed as well as demand for O₂ sparged to the vessels were compared (Table 3). The previous process utilized M20a single feed, at 36.5° C. for the culture temperature, pH 6.8, and media with a starting osmolality of 320 mOsm/kg H₂O. The new fermentation process utilized a 2 part feed, at 36.5° C. or 37° C. for the culture temperature, and a pH of 6.85 or 7.0, and culture media with a starting osmolality of 320 mOsm/kg H₂O.

TABLE 4
Comparison of two fermentation processes.

| | Previous fermentation | New fermentation |
|---|---|---|
| | process | process |
| Aggregates [%] | 6.13 | 1.31 |
| mAb final titer [g/L] | 3.12 | 7.31 |
| Highest VCN reached | 13.4 | 20.5 |
| [×10⁶ cells/mL] | | |
| Glucose solution dosed | 80.6 | 109.9 |
| [mL] | | |
| Alkali solution dosed [mL] | 0.0 | 19.7 |
| Total O₂ sparged [L] | 1284 | 5000 |

As predicted by the DoE modeling aggregation levels were significantly reduced to an acceptable level, under the optimized cell culture conditions. In addition, a greater than 2-fold increase in the final product titer was also achieved when compared to the previous base case process. Both the final aggregation level and the final titer were close to the predicted values for the optimized process (see above).

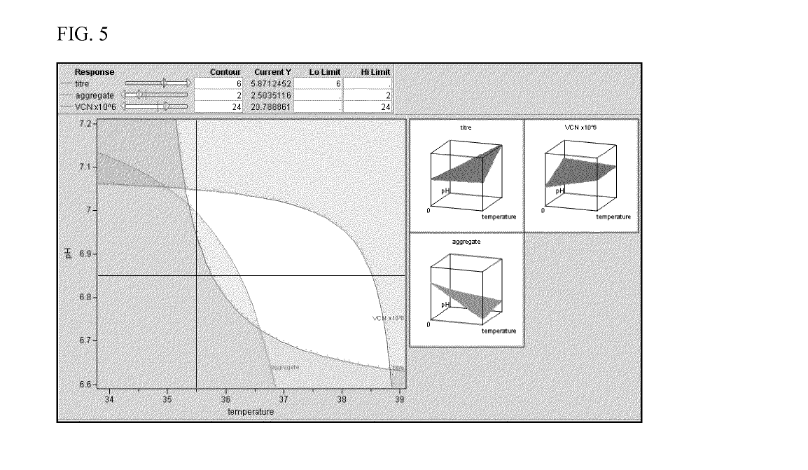

In terms of cell growth, the cells grew to higher cell densities (FIG. 4) demanding more glucose, alkali solution and oxygen under the optimized fermentation conditions when compared to the base case process. The high VCN was probably the main reason for the higher nutrient demands (i.e. oxygen, alkali and glucose solution) observed with the new optimized cell culture process. In order to identify operating conditions which could potentially lower these demands, it is possible to add VCN as a process response and constraint. An example of a DoE Contour Profiler showing the operating window for a cell culture process capable of achieving mAb aggregation levels of lower than 2% and mAb titers of greater than 6 g/L is presented in FIG. 5. An additional constraint based on the peak VCN has also been added to reflect the impact this parameter has on the higher nutrient requirements detailed previously.

By altering only three operating parameters of the fermentation process (osmolality, pH, temperature) as well as changing the type of feed, it was possible to observe differences in the mAb aggregation levels, ranging from about 1% to 6% and mAb titre, ranging from 3.1 g/L to 7.5 g/L. It has also been observed, that by changing only the type of the feed (from M20a to 2-part feed), the mAb aggregation level reduced by approximately 75% (Table 3, Conditions 11 and 12).

These data demonstrate that small changes in cell culture parameters can have a great impact on the formation of mAb aggregates. All tested factors are parameters that can be easily manipulated and this approach is therefore applicable to other cell culture processes producing therapeutic proteins.

It is still unclear what the mechanism of antibody aggregation is during the cell culture process and whether multiple mechanisms exist. However, even small changes of cell culture parameters have a great impact on formation of mAb aggregates. The identified factors can be easily manipulated to alter the solubility of proteins in aqueous solutions.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

### EMBODIMENTS

### Embodiment 1

A method of producing an anti-delta like ligand 4 (DLL4) monoclonal antibody, comprising:
culturing a mammalian cell that expresses the antibody at a temperature of about 37° C., a pH of about 7.0, and a starting osmolality of about 320 mOsm/kg H₂O,
   wherein the antibody comprises:
      a) a heavy chain variable (VH) domain as set forth in SEQ ID NO:7 and a light chain variable (VL) domain as set forth in SEQ ID NO:8; or
      b) a VH domain complementarity domain region (CDR) 1 comprising the amino acid sequence as set forth in SEQ ID NO:1, a VH domain CDR2 comprising the amino acid sequence as set forth in SEQ ID NO:2, and a VH CDR3 comprising the amino acid sequence as set forth in SEQ ID NO:3; and a VL domain CDR1 comprising the amino acid sequence as set forth in SEQ ID NO:4, a VL domain CDR2 comprising the amino acid sequence as set forth in SEQ ID NO:5 and a VL domain CDR3 comprising the amino acid sequence as set forth in SEQ ID NO:6; and
recovering the expressed anti-DLL4 antibody from the culture supernatant.

### Embodiment 2

A method of producing an anti-delta like ligand 4 (DLL4) monoclonal antibody, comprising:
culturing a mammalian cell that expresses the antibody at a temperature of about 36.5° C., a pH of about 6.85, and a starting osmolality of about 320 mOsm/kg H₂O,
   wherein the antibody comprises:
      a) a heavy chain variable (VH) domain as set forth in SEQ ID NO:7 and a light chain variable (VL) domain as set forth in SEQ ID NO:8; or
      b) a VH domain complementarity domain region (CDR) 1 comprising the amino acid sequence as set forth in SEQ ID NO:1, a VH domain CDR2 comprising the amino acid sequence as set forth in SEQ ID NO:2, and a VH CDR3 comprising the amino acid sequence as set forth in SEQ ID NO:3; and a VL domain CDR1 comprising the amino acid sequence as set forth in SEQ ID NO:4, a VL domain CDR2 comprising the amino acid sequence as set forth in SEQ ID NO:5 and a VL domain CDR3 comprising the amino acid sequence as set forth in SEQ ID NO:6; and
recovering the expressed anti-DLL4 antibody from the culture supernatant.

### Embodiment 3

The method of Embodiment 1 or 2 wherein the recovered anti-DLL4 antibody comprises less than 5% aggregate as determined by SEC-HPLC.

### Embodiment 4

The method of any of the preceding Embodiments wherein the recovered anti-DLL4 antibody comprises less than 2% aggregate as determined by SEC-H PLC.

### Embodiment 5

The method of any one of the preceding Embodiments further comprising feeding the cells with a two-part feed during the culturing.

### Embodiment 6

The method of any one of the preceding Embodiments, wherein the mammalian cell line is chosen from a Chinese Hamster Ovary (CHO), NS0, or PER.C6 cell line.

### Embodiment 7

The method of Embodiment 6, wherein the cell line is CHO.

### Embodiment 8

The method of any one of the preceding Embodiments, wherein the recovering of the anti-DLL4 antibody comprises affinity purification of the antibody.

### Embodiment 9

The method of Embodiment 8 wherein the affinity purification comprises protein A affinity chromatography.

### Embodiment 10

The method of any one of the preceding Embodiments wherein the titer of the antibody in the culture supernatant is at least 3 g/L.

### Embodiment 11

The method of any one of the preceding Embodiments wherein the titer of the antibody in the culture supernatant is at least 4 g/L.

### Embodiment 12

The method of any one of the preceding Embodiments wherein the titer of the antibody in the culture supernatant is at least 5 g/L.

### Embodiment 13

The method of any one of the preceding Embodiments wherein the titer of the antibody in the culture supernatant is at least 6 g/L.

### Embodiment 14

The method of any of the preceding Embodiments wherein the titer of the antibody in the culture supernatant is at least 7 g/L.

### Embodiment 15

The method of any of the preceding Embodiments wherein the anti-DLL4 antibody comprises VH domain as set forth in SEQ ID NO:7 and a VL domain as set forth in SEQ ID NO:8.

### Embodiment 16

The method of any of Embodiments 1-14 wherein the anti-DLL4 antibody comprises a VH domain CDR1 comprising the amino acid sequence as set forth in SEQ ID NO:1, a VH domain CDR2 comprising the amino acid sequence as set forth in SEQ ID NO:2, and a VH CDR3 comprising the amino acid sequence as set forth in SEQ ID NO:3; and a VL domain CDR1 comprising the amino acid sequence as set forth in SEQ ID NO:4, a VL domain CDR2 comprising the amino acid sequence as set forth in SEQ ID NO:5 and a VL domain CDR3 comprising the amino acid sequence as set forth in SEQ ID NO:6

### Embodiment 17

A method of reducing aggregate content in a protein A-purified monoclonal antibody product to less than about 5%, the method comprising:
a) culturing a mammalian cell line that expresses the antibody in a culture medium having a starting osmolality of about 320 mOsm/kg H₂O, and at a temperature of about 37° C., and a pH of about 7.0;
   wherein the cell line expresses an anti-DLL4 antibody comprising a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO: 3, a VL CDR1 comprising the amino acid sequence of SEQ ID NO:4, a VL CDR2 comprising the amino acid sequence of SEQ ID NO:5, and a VL CDR3 comprising an amino acid sequence of SEQ ID NO: 6; and
   wherein the culture process comprises using a two part feed to feed the cells;
b) recovering the expressed antibody from the culture supernatant; and
c) purifying the expressed antibody using affinity chromatography.

### Embodiment 18

A method of reducing aggregate content in a protein A-purified monoclonal antibody product to less than about 5%, the method comprising:
a) culturing a mammalian cell line that expresses the antibody in a culture medium having a starting osmolality of about 320 mOsm/kg H₂O, and at a temperature of about 36.5° C., and a pH of about 6.85;
   wherein the cell line expresses an anti-DLL4 antibody comprising a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO: 3, a VL CDR1 comprising the amino acid sequence of SEQ ID NO:4, a VL CDR2 comprising the amino acid sequence of SEQ ID NO:5, and a VL CDR3 comprising an amino acid sequence of SEQ ID NO: 6; and
   wherein the culture process comprises using a two part feed to feed the cells;
b) recovering the expressed antibody from the culture supernatant; and
c) purifying the expressed antibody using affinity chromatography.

### Embodiment 19

The method of Embodiment 17 or 18 wherein the affinity chromatography comprises protein A affinity chromatography.

### Embodiment 20

The method of any of Embodiment 17-19 wherein the mammalian cell is a CHO cell.

### Embodiment 21

The method of any of Embodiments 17-20 wherein the antibody comprises a VH domain comprising the amino acid sequence of SEQ ID NO:7 and a VL domain comprising the amino acid sequence of SEQ ID NO:8.

### Embodiment 22

The method of any of Embodiments 17-21 wherein the purified anti-DLL4 antibody comprises less than 2% aggregate as determined by SEC-HPLC.

### Embodiment 23

The method of any one of Embodiments 17-22 wherein the titer of the antibody in the culture supernatant is at least 3 g/L.

### Embodiment 24

The method of any one of Embodiments 17-23 wherein the titer of the antibody in the culture supernatant is at least 4 g/L.

### Embodiment 25

The method of any one of Embodiments 17-24 wherein the titer of the antibody in the culture supernatant is at least 5 g/L.

### Embodiment 26

The method of any one of Embodiments 17-25 wherein the titer of the antibody in the culture supernatant is at least 6 g/L.

### Embodiment 27

The method of any of Embodiments 17-26 wherein the titer of the antibody in the culture supernatant is at least 7 g/L.

### Embodiment 28

A method of reducing aggregates of an anti-DLL4 monoclonal antibody (mAb) comprising culturing a CHO cell that secretes the anti-DLL4 mAb under conditions of temperature, pH, and osmolality, that produce less aggregate than culture of the same mAb-producing CHO cell under conditions comprising a temperature of 36.5° C., a pH of 6.8, and starting osmolality of 320 mOsm/kg H₂O in a bioreactor using a single feed,
wherein the anti-DLL4 antibody comprises a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO: 3, a VL CDR1 comprising the amino acid sequence of SEQ ID NO:4, a VL CDR2 comprising the amino acid sequence of SEQ ID NO:5, and a VL CDR3 comprising an amino acid sequence of SEQ ID NO: 6.

### Embodiment 29

The method of Embodiment 28 wherein the conditions that produce less aggregate comprise one of:
a) pH 7.0, temperature 34° C., and starting osmolality 400 mOsm/kg H₂O; or
b) pH 6.85, temperature 35.5° C., and starting osmolality 360 mOsm/kg H₂O; or
c) pH 6.7, temperature 37° C., and starting osmolality 400 mOsm/kg H₂O; or
d) pH 6.7, temperature 34° C., and starting osmolality 320 mOsm/kg H₂O; or
e) pH 7.0, temperature 37° C., and starting osmolality 320 mOsm/kg H₂O; or
f) pH 7.0, temperature 37° C., and starting osmolality 400 mOsm/kg H₂O; or
g) pH 6.85, temperature 35.5° C., and starting osmolality 360 mOsm/kg H₂O; or
h) pH 7.0, temperature 34° C., and starting osmolality 320 mOsm/kg H₂O; or
i) pH 6.7, temperature 37° C., and starting osmolality 320 mOsm/kg H₂O; or
j) pH6.85, temperature 36.5° C., and starting osmolality 320 mOsm/kg H₂O.

### Embodiment 30

The method of Embodiment 28 or 29 wherein the culturing further comprises feeding the cells with a two part feed.

### Embodiment 31

The method of any of Embodiments 28-30 wherein the anti-DLL4 antibody comprises a VH domain comprising the amino acid sequence as shown in SEQ ID NO:7 and a VL domain comprising the amino acid sequence as shown in SEQ ID NO:8.

### Embodiment 32

An antibody composition produced by any of the preceding embodiments, wherein the antibody composition comprises less than about 1.4% aggregate, as determined by SEC-HPLC.

### REFERENCES

Aggarwal, S. (2010). What's fuelling the biotech engine—2009-2010? Nature Biotechnology, 28 (11), 1165-1171.
Arakawa, T. et al. (2006). Aggregation analysis of therapeutic proteins, Part 1. BioProcess International, 4 (10), 42-43.
Barnard, J. G. et al. (2010). Subvisible particle counting provides a sensitive method of detecting and quantifying aggregation of monoclonal antibody caused by freeze-thawing: insights into the roles of particles in the protein aggregation pathway. Journal of Pharmaceutical Sciences, 100 (2), 492-503.
Chen, S. et al. (2010). The use of native cation-exchange chromatography to study aggregation and phase separation of monoclonal antibodies. Protein Science, 19, 1191-1204.
Cromwell, M. E. M. et al. (2006). Protein aggregation and bioprocessing. The AAPS Journal, 8 (3), E572-E579.
Franco, R. et al. (1999). Influence of osmolarity and pH increase to achieve a reduction of monoclonal antibodies aggregates in a production process. Cytotechnology, 29, 11-25.
Gabrielson, J. P. et al. (2006). Quantitation of aggregate levels in a recombinant humanized monoclonal antibody formulation by Size-Exclusion Chromatography, Asymmetrical Flow Field Flow Fractionation, and Sedimentation Velocity. Journal of Pharmaceutical Sciences, 96 (2), 268-279.
Hacker, D. L. et al. (2009). 25 years of recombinant proteins from reactor-grown cells—Where do we go from here? Biotechnology Advances, 27, 1023-1017.
Hawe, A. et al. (2009). Structural properties of monoclonal antibody aggregates induced by freeze-thawing and thermal stress. European Journal of Pharmaceutical Sciences, 38, 79-87.
Jain, E. & Kumar, A. (2008). Upstream processes in antibody production: Evaluation of critical parameters. Biotechnology Advances, 26, 46-72.
Liu, H. et al. (2009). Analysis of reduced monoclonal antibodies using size exclusion chromatography coupled with mass spectrometry. Journal of The American Society for Mass Spectrometry, 20, 2258-2264.
Mahler, H. C. et al. (2008). Protein aggregation: pathways, induction factors and analysis. Journal of Pharmaceutical Sciences, 98 (9), 2909-2934.
Pan, H. et al. (2009). Quantitation of soluble aggregates in recombinant monoclonal antibody cell culture by pH-gradient protein A chromatography. Analytical Biochemistry, 388, 273-278.
Philo, J. S. & Arakawa, T. (2009). Mechanisms of protein aggregation. Current Pharmaceutical Biotechnology, 10, 348-351.
Shellekens, H. (2005). Factors influencing the immunogenicity of therapeutic proteins. Nephrology Dialysis Transplantation, 20, vi3-vi9.
Shukla, A. A. et al. (2007). Protein aggregation kinetics during Protein A chromatography: Case study for an Fc fusion protein. Journal of Chromatography A, 1171 (1-2), 22-28.
Shukla, A. A & Thommes, J. (2010). Recent advances in large-scale production of monoclonal antibodies and related proteins. Trends in Biotechnology, 28 (5), 253-261.
Rathore, A. S. & Winkle, H. (2009). Quality by design for biopharmaceuticals. Nature Biotechnology, 27, 26-34.
Rosenberg, A. S. (2006). Effects of protein aggregates: An immunologic perspective. The AAPS Journal, 8 (3), E501-E507.
Wang, W. et al. (2010). Protein aggregation—pathways and influencing factors. International Journal of Pharmaceuticals, 390, 89-99.

What is claimed is:

1. A method of producing an anti-delta like ligand 4 (DLL4) monoclonal antibody, comprising:
culturing a mammalian cell that expresses the antibody at a temperature of about 37° C., a pH of about 7.0, and a starting osmolality of about 320 mOsm/kg H₂O,
wherein the antibody comprises:
a) a heavy chain variable (VH) domain as set forth in SEQ ID NO:7 and a light chain variable (VL) domain as set forth in SEQ ID NO:8; or
b) a VH domain complementarity domain region (CDR) 1 comprising the amino acid sequence as set forth in SEQ ID NO:1, a VH domain CDR2 comprising the amino acid sequence as set forth in SEQ ID NO:2, and a VH CDR3 comprising the amino acid sequence as set forth in SEQ ID NO:3; and a VL domain CDR1 comprising the amino acid sequence as set forth in SEQ ID NO:4, a VL domain CDR2 comprising the amino acid sequence as set forth in SEQ ID NO:5 and a VL domain CDR3 comprising the amino acid sequence as set forth in SEQ ID NO:6; and
recovering the expressed anti-DLL4 antibody from the culture supernatant.

2. A method of producing an anti-delta like ligand 4 (DLL4) monoclonal antibody, comprising:
culturing a mammalian cell that expresses the antibody at a temperature of about 36.5° C., a pH of about 6.85, and a starting osmolality of about 320 mOsm/kg H₂O,
wherein the antibody comprises:
a) a heavy chain variable (VH) domain as set forth in SEQ ID NO:7 and a light chain variable (VL) domain as set forth in SEQ ID NO:8; or
b) a VH domain complementarity domain region (CDR) 1 comprising the amino acid sequence as set forth in SEQ ID NO:1, a VH domain CDR2 comprising the amino acid sequence as set forth in SEQ ID NO:2, and a VH CDR3 comprising the amino acid sequence as set forth in SEQ ID NO:3; and a VL domain CDR1 comprising the amino acid sequence as set forth in SEQ ID NO:4, a VL domain CDR2 comprising the amino acid sequence as set forth in SEQ ID NO:5 and a VL domain CDR3 comprising the amino acid sequence as set forth in SEQ ID NO:6; and
recovering the expressed anti-DLL4 antibody from the culture supernatant.

3. The method of claim 1 or 2 wherein the recovered anti-DLL4 antibody comprises less than 5% aggregate as determined by SEC-HPLC.

4. The method of any of the preceding claims wherein the recovered anti-DLL4 antibody comprises less than 2% aggregate as determined by SEC-HPLC.

5. The method of any one of the preceding claims further comprising feeding the cells with a two-part feed during the culturing.

6. The method of any one of the preceding claims, wherein the mammalian cell line is chosen from a Chinese Hamster Ovary (CHO), NS0, or PER.C6 cell line.

7. The method of claim 6, wherein the cell line is CHO.

8. The method of any one of the preceding claims, wherein the recovering of the anti-DLL4 antibody comprises affinity purification of the antibody.

9. The method of claim 6 wherein the affinity purification comprises protein A affinity chromatography.

10. The method of any one of the preceding claims wherein the titer of the antibody in the culture supernatant is at least 3 g/L.

11. The method of any one of the preceding claims wherein the titer of the antibody in the culture supernatant is at least 4 g/L.

12. The method of any one of the preceding claims wherein the titer of the antibody in the culture supernatant is at least 5 g/L.

13. The method of any one of the preceding claims wherein the titer of the antibody in the culture supernatant is at least 6 g/L.

14. The method of any of the preceding claims wherein the titer of the antibody in the culture supernatant is at least 7 g/L.

15. The method of any of the preceding claims wherein the anti-DLL4 antibody comprises VH domain as set forth in SEQ ID NO:7 and a VL domain as set forth in SEQ ID NO:8.

16. The method of any of claims 1-14 wherein the anti-DLL4 antibody comprises a VH domain CDR1 comprising the amino acid sequence as set forth in SEQ ID NO:1, a VH domain CDR2 comprising the amino acid sequence as set forth in SEQ ID NO:2, and a VH CDR3 comprising the amino acid sequence as set forth in SEQ ID NO:3; and a VL domain CDR1 comprising the amino acid sequence as set forth in SEQ ID NO:4, a VL domain CDR2 comprising the amino acid sequence as set forth in SEQ ID NO:5 and a VL domain CDR3 comprising the amino acid sequence as set forth in SEQ ID NO:6

17. A method of reducing aggregate content in a protein A-purified monoclonal antibody product to less than about 5%, the method comprising:
a) culturing a mammalian cell line that expresses the antibody in a culture medium having a starting osmolality of about 320 mOsm/kg H₂O, and at a temperature of about 37° C., and a pH of about 7.0;
wherein the cell line expresses an anti-DLL4 antibody comprising a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO: 3, a VL CDR1 comprising the amino acid sequence of SEQ ID NO:4, a VL CDR2 comprising the amino acid sequence of SEQ ID NO:5, and a VL CDR3 comprising an amino acid sequence of SEQ ID NO: 6; and
wherein the culture process comprises using a two part feed to feed the cells;
b) recovering the expressed antibody from the culture supernatant; and
c) purifying the expressed antibody using affinity chromatography.

18. A method of reducing aggregate content in a protein A-purified monoclonal antibody product to less than about 5%, the method comprising:
a) culturing a mammalian cell line that expresses the antibody in a culture medium having a starting osmolality of about 320 mOsm/kg H₂O, and at a temperature of about 36.5° C., and a pH of about 6.85;
wherein the cell line expresses an anti-DLL4 antibody comprising a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO: 3, a VL CDR1 comprising the amino acid sequence of SEQ ID NO:4, a VL CDR2 comprising the amino acid sequence of SEQ ID NO:5, and a VL CDR3 comprising an amino acid sequence of SEQ ID NO: 6; and
wherein the culture process comprises using a two part feed to feed the cells;
b) recovering the expressed antibody from the culture supernatant; and
c) purifying the expressed antibody using affinity chromatography.

19. The method of claim 17 or 18 wherein the affinity chromatography comprises protein A affinity chromatography.

20. The method of any of claims 17-19 wherein the mammalian cell is a CHO cell.

21. The method of any of claims 17-20 wherein the antibody comprises a VH domain comprising the amino acid sequence of SEQ ID NO:7 and a VL domain comprising the amino acid sequence of SEQ ID NO:8.

22. The method of any of claims 17-21 wherein the purified anti-DLL4 antibody comprises less than 2% aggregate as determined by SEC-HPLC.

23. The method of any one of claims 17-22 wherein the titer of the antibody in the culture supernatant is at least 3 g/L.

24. The method of any one of claims 17-23 wherein the titer of the antibody in the culture supernatant is at least 4 g/L.

25. The method of any one of claims 17-24 wherein the titer of the antibody in the culture supernatant is at least 5 g/L.

26. The method of any one of claims 17-25 wherein the titer of the antibody in the culture supernatant is at least 6 g/L.

27. The method of any of claims 17-26 wherein the titer of the antibody in the culture supernatant is at least 7 g/L.

28. A method of reducing aggregates of an anti-DLL4 monoclonal antibody (mAb) comprising culturing a CHO cell that secretes the anti-DLL4 mAb under conditions of temperature, pH, and osmolality, that produce less aggregate than culture of the same mAb-producing CHO cell under conditions comprising a temperature of 36.5° C., a pH of 6.8, and starting osmolality of 320 mOsm/kg H₂O in a bioreactor
wherein the anti-DLL4 antibody comprises a VH CDR1 comprising the amino acid sequence of SEQ ID NO:1, a VH CDR2 comprising the amino acid sequence of SEQ ID NO:2, a VH CDR3 comprising the amino acid sequence of SEQ ID NO: 3, a VL CDR1 comprising the amino acid sequence of SEQ ID NO:4, a VL CDR2 comprising the amino acid sequence of SEQ ID NO:5, and a VL CDR3 comprising an amino acid sequence of SEQ ID NO: 6.

29. The method of claim 28 wherein the conditions that produce less aggregate comprise one of:
a) pH 7.0, temperature 34° C., and starting osmolality 400 mOsm/kg H₂O; or
b) pH 6.85, temperature 35.5° C., and starting osmolality 360 mOsm/kg H₂O; or
c) pH 6.7, temperature 37° C., and starting osmolality 400 mOsm/kg H₂O; or
d) pH 6.7, temperature 34° C., and starting osmolality 320 mOsm/kg H₂O; or
e) pH 7.0, temperature 37° C., and starting osmolality 320 mOsm/kg H₂O; or
f) pH 7.0, temperature 37° C., and starting osmolality 400 mOsm/kg H₂O; or
g) pH 6.85, temperature 35.5° C., and starting osmolality 360 mOsm/kg H₂O; or
h) pH 7.0, temperature 34° C., and starting osmolality 320 mOsm/kg H₂O; or
i) pH 6.7, temperature 37° C., and starting osmolality 320 mOsm/kg H₂O; or
j) pH6.85, temperature 36.5° C., and starting osmolality 320 mOsm/kg H₂O.

30. The method of claim 28 or 29 wherein the culturing further comprises feeding the cells with a two part feed.

31. The method of any of claims 28-30 wherein the anti-DLL4 antibody comprises a VH domain comprising the amino acid sequence as shown in SEQ ID NO:7 and a VL domain comprising the amino acid sequence as shown in SEQ ID NO:8.

32. An antibody composition produced by any of the preceding claims, wherein the antibody composition comprises less than about 1.4% aggregate, as determined by SEC-H PLC.
